(19) United States
(12) Patent Application Publication
(10) Pub. No.: US 2022/0340517 A1
(43) Pub. Date: Oct. 27, 2022

(54) Continuous synthesis method for ethoxymethylenemalononitrile

(71) Applicant: ASYMCHEM LABORATORIES (TIANJIN) CO., LTD., Tianjin (CN)

(72) Inventors: Hao HONG, Morrisville, NC (US); Jiangping LU, Tianjin (CN); Denghui BAO, Tianjin (CN); Chaojie LIU, Tianjin (CN); Xiaotong JIA, Tianjin (CN)

(21) Appl. No.: 17/772,660

(22) PCT Filed: Dec. 18, 2019
(86) PCT No.: PCT/CN2019/126378

§ 371 Date: Apr. 28, 2022

(51) Int. Cl. C07C 255/15

(52) U.S. Cl. CPC C07C 255/15

(57) ABSTRACT

Provided is a continuous synthesis method for ethoxymethylenemalononitrile. The method includes the following steps: malononitrile, triethyl orthoformate and acetic anhydride are continuously fed into a continuous reaction device to perform a condensation reaction, to obtain the ethoxymethylenemalononitrile, and in the process of the condensation reaction, the generated ethoxymethylenemalononitrile is continuously discharged; herein, the molar ratio of the malononitrile, the triethyl orthoformate and the acetic anhydride is 1:(0.9-6.0):(2.0-6.0). By adopting the continuous reaction device in the present disclosure, since the amount of materials involved in the reaction per unit time is greatly reduced, a high temperature dangerous area is reduced, and a safety risk is greatly reduced. In addition, through the continuous reactor, the raw materials may be transiently heated to the reaction temperature, so that the decomposition of the raw materials caused by the long-time heating process is avoided, and the yield is significantly improved. Moreover, in the reaction process of the present disclosure, there is no need for a second-class toxic solvent such as a toluene.

### TECHNICAL FIELD

The present disclosure relates to the technical field of organic synthesis, in particular to a continuous synthesis method for ethoxymethylenemalononitrile.

### BACKGROUND

Ethoxymethylenemalononitrile is white to off-white crystalline powder, insoluble in water, and soluble in methanol, ethanol and other organic solvents. The melting point is 65˜67° C., and the boiling point is 160° C. (12 mmHg). It is commonly used as an intermediate in preparation of industrial chemicals and pharmaceutical chemicals, such as an intermediate for preparation of milrinone. Therefore, the ethoxymethylenemalononitrile has an extremely wide field and a very broad development prospect, and it is of the great significance to develop a high-efficiency and low-cost synthesis process of the ethoxymethylenemalononitrile.

At present, a preparation method for the ethoxymethylenemalononitrile includes the following steps: in the presence of an organic solvent and a catalyst, triethyl orthoformate and malononitrile are refluxed until a reaction is completed, herein the organic solvent is usually a high-boiling-point toxic solvent such as a toluene. The toluene is a second-class solvent restricted for use according to regulations of the International Council for Harmonisation of Technical Requirements for Pharmaceuticals for Human Use (ICH).

Liu Qiming et al. (Liu Qiming, Su Yuyong, Chen Bangyin, and Zhang Hanping. Synthesis and structural identification of phosphodiesterase inhibitor milrinone [J]. Journal of Huazhong University of Science and Technology (medical edition), 2005, 34(1): 74-75) disclose a preparation method for ethoxymethylenemalononitrile, this method uses a large amount of the toxic toluene as a solvent, and a catalyst zinc chloride used is not dissolved in the organic solvent (toluene) and is difficult to fully contact with a reaction substance, so that the catalytic effect is not ideal, the reaction speed and reaction effect are affected, and the yield is only 79% (calculated in malononitrile), so there are defects such as unfavorable to environmental protection, high production cost, difficult solvent recovery, and low yield.

A Chinese patent (CN102584626) provides a method for preparing ethoxymethylenemalononitrile, this method uses malononitrile and triethyl orthoformate as initial raw materials, and uses the zinc chloride as a catalyst. The catalyst zinc chloride used is not dissolved in the organic solvent, and is difficult to fully contact with the reaction substance, so that the catalytic effect is not ideal, the reaction speed and reaction effect are affected, the amount of the triethyl orthoformate used is relatively large, and the yield is only 85% (calculated in malononitrile). The method still has problems of low yield, high cost, slow reaction speed and low production efficiency.

In addition, a foreign patent (WO2014/2111, 2014) provides a new method for preparing ethoxymethylenemalononitrile, acetic anhydride is used as a reagent for a reaction, and the reaction temperature is 110˜140° C., but the safe operation temperature suggested by a process is T<107° C. (Td24), the Stoessel grade of the reaction is 5, and the danger level is high. The use of a traditional tank reactor for scale-up production has a higher safety risk, and the large-scale production may not be performed.

Based on the above reasons, it is necessary to provide an ethoxymethylenemalononitrile production process with safer operation, less pollution and high yield.

### SUMMARY

A main purpose of the present disclosure is to provide a continuous synthesis method for ethoxymethylenemalononitrile, as to solve problems in an existing technology that the yield is low, the environment is polluted by a toxic solvent and the safety is poor while the ethoxymethylenemalononitrile is prepared.

In order to achieve the above purpose, according to one aspect of the present disclosure, a continuous synthesis method for ethoxymethylenemalononitrile is provided, and it includes the following steps: malononitrile, triethyl orthoformate and acetic anhydride are continuously fed into a continuous reaction device to perform a condensation reaction, to obtain the ethoxymethylenemalononitrile, and in the process of the condensation reaction, the generated ethoxymethylenemalononitrile is continuously discharged; herein, the molar ratio of the malononitrile, the triethyl orthoformate and the acetic anhydride is 1:(0.9-6.0):(2.0-6.0).

Further, the molar ratio of the malononitrile, the triethyl orthoformate and the acetic anhydride is 1:(1.1-1.5):(2.1-2.5).

Further, the reaction temperature of the condensation reaction is 110 to 150° C., and the reaction pressure is 0.3 to 10 MPa.

Further, the reaction temperature of the condensation reaction is 110 to 120° C., and the reaction pressure is 0.3 to 10 MPa.

Further, the condensation reaction is carried out without or with a solvent.

Further, in addition, while the solvent is used, the solvent is selected from one or more of ethyl acetate, tetrahydrofuran, 2-methyltetrahydrofuran, 1,4-dioxane, ethylene glycol dimethyl ether, diethylene glycol dimethyl ether, benzene, toluene, xylene, acetone, acetonitrile, methanol, ethanol, isopropanol, and ethylene glycol.

Further, during the condensation reaction, the residence time of the malononitrile, the triethyl orthoformate and the acetic anhydride in the continuous reaction device is 30 to 400 min.

Further, in the process of the condensation reaction, the residence time of the malononitrile, the triethyl orthoformate and the acetic anhydride in the continuous reaction device is 90 to 120 min.

Further, the continuous reaction device is a tubular reactor or a columnar reactor.

Further, in the process of the condensation reaction, the malononitrile, the triethyl orthoformate, the acetic anhydride and the optional solvent are pumped into the continuous reaction device, and the total flowrate is 20 ml/min to 5 L/min.

The present disclosure achieves the continuous production of the ethoxymethylenemalononitrile by adopting the continuous reaction device and the continuous condensation reaction of the malononitrile, the triethyl orthoformate and the acetic anhydride. Compared with a traditional kettle-type reaction, since the amount of materials involved in the reaction per unit time is greatly reduced, a high temperature dangerous area is reduced, and a safety risk is greatly reduced. In addition, through the continuous reactor, the raw materials may be transiently heated to the reaction temperature, so that the decomposition of the raw materials caused by the long-time heating process is avoided, and the yield is significantly improved. Moreover, in the reaction process of the present disclosure, there is no need for a second-class toxic solvent such as a toluene. In addition, since the use of the continuous processes and continuous reaction device may significantly reduce the safety risk of the process, the scale-up production may be achieved.

### DETAILED DESCRIPTION OF THE EMBODIMENTS

It should be noted that embodiments in the present application and features of the embodiments may be combined with each other in the case without conflicting. The present disclosure is described in detail below with reference to the embodiments.

As described in the background section, there are problems in an existing technology that the yield is low, the environment is polluted by a toxic solvent, and the safety is poor while ethoxymethylenemalononitrile is prepared.

In order to solve the above problems, the present disclosure provides a continuous synthesis method for ethoxymethylenemalononitrile, and it includes the following steps: malononitrile, triethyl orthoformate and acetic anhydride are continuously fed into a continuous reaction device to perform a condensation reaction, to obtain the ethoxymethylenemalononitrile, and in the process of the condensation reaction, the generated ethoxymethylenemalononitrile is continuously discharged; herein, the molar ratio of the malononitrile, the triethyl orthoformate and the acetic anhydride is 1:(0.9-6.0):(2.0-6.0).

The present disclosure achieves the continuous production of the ethoxymethylenemalononitrile by adopting the continuous reaction device and the continuous condensation reaction of the malononitrile, the triethyl orthoformate and the acetic anhydride. Compared with a traditional kettle-type reaction, since the amount of materials involved in the reaction per unit time is greatly reduced, a high temperature dangerous area is reduced, and a safety risk is greatly reduced. In addition, through the continuous reactor, the raw materials may be transiently heated to the reaction temperature, so that the decomposition of the raw materials caused by the long-time heating process is avoided, and the yield is significantly improved (the highest yield may reach more than 98.5%). Moreover, in the reaction process of the present disclosure, there is no need for a second-class toxic solvent such as a toluene. In addition, since the use of the continuous processes and continuous reaction device may significantly reduce the safety risk of the process, the scale-up production may be achieved.

In addition to the above beneficial effects, compared with a traditional kettle-type production process, the present disclosure also has the advantages of low cost and small amount of three wastes and the like, and because the continuous reaction greatly shortens the reaction time, the production efficiency is significantly improved. At the same time, the post-treatment is simple, and the product purity is high, and even reaches 99.5% or more.

In order to further improve the reaction efficiency and improve the product yield, in a preferred embodiment, the molar ratio of the malononitrile, the triethyl orthoformate and the acetic anhydride is 1:(1.1-1.5):(2.1-2.5).

As described above, the raw materials may be transiently heated to the reaction temperature through the continuous reactor, so that the decomposition of the raw materials caused by the long-time heating process is avoided, and the yield is significantly improved. In order to further improve the reaction efficiency and product yield, and avoid the decomposition of the raw materials caused by the excessive temperature at the same time, in a preferred embodiment, the reaction temperature of the condensation reaction is 110-150° C., and the reaction pressure is 0.3-10 MPa. More preferably, the reaction temperature of the condensation reaction is 110-120° C., and the reaction pressure is 0.3-10 MPa.

Because the safety of the continuous condensation reaction is high, the above malononitrile, triethyl orthoformate and acetic anhydride may be directly reacted in a solvent-free state. Certainly, the reaction may also be performed in the presence of solvents, and these solvents are non-second-class toxic solvents. Preferably, in addition while the solvent is used, the solvent is selected from one or more of ethyl acetate, tetrahydrofuran, 2-methyltetrahydrofuran, 1,4-dioxane, ethylene glycol dimethyl ether, diethylene glycol dimethyl ether, benzene, toluene, xylene, acetone, acetonitrile, methanol, ethanol, isopropanol, and ethylene glycol. Preferably, the amount of the solvent relative to the malononitrile is 1-50 mg/g.

In a preferred embodiment, in the process of the condensation reaction, the residence time of the malononitrile, the triethyl orthoformate and the acetic anhydride in the continuous reaction device is 30-400 min, more preferably 90-120 min. Under this residence time, the condensation reaction is more sufficient, and it is beneficial to further improve the reaction yield.

In a preferred embodiment, in the process of the condensation reaction, the malononitrile, the triethyl orthoformate, the acetic anhydride and the optional solvent are pumped into the continuous reaction device by a pump, and the total flowrate is 20 mL/min to 5 L/min, preferably 100 to 300 mL/min. It should be noted that the above pumping speed refers to the total pumping speed of all the raw materials, and those skilled in the art may convert the feeding speed of each raw material according to the usage ratio of each raw material required, it is not repeatedly described here.

There are various specific liquid feeding modes: Mode I, under a solvent-free condition, the malononitrile and acetic anhydride are mixed to form a raw material A, and the triethyl orthoformate is used as a raw material B, the raw material A and the raw material B are simultaneously pumped into the continuous reactor for reaction through a pump A and a pump B respectively; and Mode II, while the solvent is involved, the malononitrile and acetic anhydride compounds are dissolved in a part of the solvent to form a raw material A, and the triethyl orthoformate is dissolved in another part of the solvent, to form a raw material B, and then the raw material A and the raw material B are simultaneously pumped into the continuous reactor for reaction through the pump A and the pump B respectively.

The above continuous reactor may preferably be a tubular reactor or a columnar reactor. More preferably, the continuous reactor is a coil-type reactor, and the internal special coil structure thereof is helpful to increase the reaction effect. More preferably, the continuous reactor includes a coil, a heat exchange device, a temperature detection device, a pressure detection device, a liquid flow controller, an online portable appliance testing (PAT) device, and an automated control system; the coil is used to provide a reaction site; the heat exchange device is used to adjust the temperature inside the coil; the temperature detection device is used to monitor the reaction temperature in the coil; the pressure detection device is used to monitor the reaction pressure in the coil; the liquid flow controller is connected between the pump and a feeding port of the coil, and used to adjust and control the feeding amount; the online PAT device is used to detect the product composition in the coil; the automated control system is electrically connected with the pump, the liquid flow controller, the heat exchange device, the temperature detection device, the pressure detection device and the online PAT device. The automated control system is used, and relevant parameters of the reaction, such as the temperature, the pressure, and the flow rate, may be precisely controlled and fed back in real time.

After the generated ethoxymethylenemalononitrile is continuously discharged, the above synthesis method also includes a step of purifying it, preferably, the purification step includes: a discharged product is vacuum-concentrated, and then n-heptane is dropwise added to a system so that the ethoxymethylenemalononitrile is precipitated, and it is filtered, to obtain an ethoxymethylenemalononitrile product.

The present application is further described in detail below with reference to the specific embodiments, and these embodiments should not be construed as limiting a scope of protection claimed by the present application.

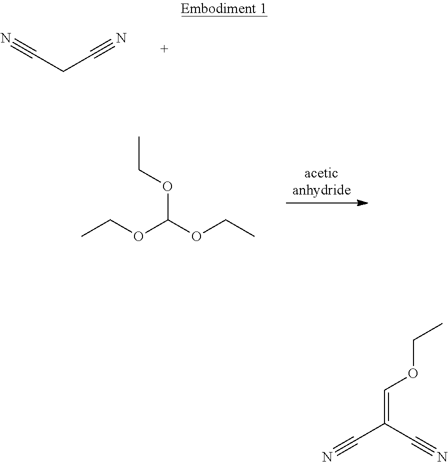

The feeding amount is 1.0 kg, and malononitrile (1.0 kg, 15.14 mol) and acetic anhydride (3.25 kg, 31.79 mol) are mixed uniformly at a room temperature, and marked as a material A; triethyl orthoformate (2.47 kg, 16.65 mol) is marked as a material B. The speed of a pump A is: 248 g/min, the speed of a pump B is: 157 g/min, the material A and the material B are respectively pumped into a continuous pipeline reactor (the volume is 50 L) by the pumps, the temperature is controlled at 120° C., the outlet back pressure of the continuous reactor is 0.3-0.5 MPa, the residence time is 100 min, the outlet sampling and gas chromatography (GC) analysis are performed, and the remaining raw material is 0.0-0.5%. A system flowing out from the continuous reactor is concentrated to 6 V under vacuum, the temperature is controlled at 20-30° C., and 4 V of n-heptane is dropwise added to the above system, after being dropwise added, the system precipitates a large amount of a solid. After being filtered, the solid is dried to obtain 1.83 kg of an ethoxymethylenemalononitrile product, the GC purity is 99.7%, and the yield is 98.69%.

### Embodiment 2

A pump A system is malononitrile (100 g, 1.51 mol)+acetic anhydride (325 g, 3.18 mol), a pump B system is triethyl orthoformate (247 g, 1.67 mol), the speed of a pump A is: 24.8 g/min, and the speed of a pump B is: 15.7 g/min. The pump A and the pump B are simultaneously opened to pump two raw materials into a continuous reactor (the volume is 5 L), the temperature is controlled at 130° C., the outlet back pressure of the continuous reactor is 0.3-0.5 MPa, the residence time is 100 min, the outlet sampling and GC analysis are performed, and the remaining raw material is 0.0-0.5%. An effluent system is concentrated to 6 V, and 4 V of n-heptane is dropwise added for crystallization. After being filtered, 165.0 g of an ethoxymethylenemalononitrile product is obtained, the GC purity is 97.2%, and the yield is 79.8%.

A difference from Embodiment 1 is that: the reaction temperature is 130° C.

### Embodiment 3

A pump A system is malononitrile (100 g, 1.51 mol)+acetic anhydride (325 g, 3.18 mol)+ethanol (100 ml), a pump B system is triethyl orthoformate (247 g, 1.67 mol)+ethanol (100 ml), the speed of a pump A is: 24.8 g/min, and the speed of a pump B is: 15.7 g/min. The pump A and the pump B are simultaneously opened to pump two raw materials into a continuous reactor (the volume is 5 L), the temperature is controlled at 120° C., the outlet back pressure of the continuous reactor is 0.3-0.5 MPa, the residence time is 100 min, the outlet sampling and GC analysis are performed, and the remaining raw material is 0.0-0.5%. An effluent system is concentrated to 6 V, and 4 V of n-heptane is dropwise added for crystallization. After being filtered, 175.0 g of an ethoxymethylenemalononitrile product is obtained, the GC purity is 75.3%, and the yield is 66.15%.

A difference from Embodiment 1 is that: the ethanol is used as a solvent.

### Embodiment 4

A pump A system is malononitrile (100 g, 1.51 mol)+acetic anhydride (618.14 g, 6.05 mol), a pump B system is triethyl orthoformate (201.9 g, 1.36 mol), the speed of a pump A is: 24.8 g/min, and the speed of a pump B is: 15.7 g/min. The pump A and the pump B are simultaneously opened to pump two raw materials into a continuous reactor (the volume is 5 L), the temperature is controlled at 120° C., the outlet back pressure of the continuous reactor is 0.3-0.5 MPa, the residence time is 100 min, the outlet sampling and GC analysis are performed, and the remaining raw material is 1.6-2.5%. An effluent system is concentrated to 6 V, and 4 V of n-heptane is dropwise added for crystallization. After being filtered, 173.0 g of an ethoxymethylenemalononitrile product is obtained, the gas phase purity is 91.1%, and the yield is 83.7%.

A difference from Embodiment 1 is that: malononitrile:triethyl orthoformate:acetic anhydride=1:0.9:4.0 in molar ratio.

### Embodiment 5

A pump A system is malononitrile (100 g, 1.51 mol)+acetic anhydride (386.3 g, 3.78 mol), a pump B system is triethyl orthoformate (336.5 g, 2.27 mol), the speed of a pump A is: 24.8 g/min, and the speed of a pump B is: 15.7 g/min. The pump A and the pump B are simultaneously opened to pump two raw materials into a continuous reactor (the volume is 5 L), the temperature is controlled at 120° C., the outlet back pressure of the continuous reactor is 0.3-0.5 MPa, the residence time is 100 min, the outlet sampling and GC analysis are performed, and the remaining raw material is 0.0-0.5%. An effluent system is concentrated to 6 V, and 4 V of n-heptane is dropwise added for crystallization. After being filtered, 181.0 g of an ethoxymethylenemalononitrile product is obtained, the GC purity is 99.6%, and the yield is 98.52%.

A difference from Embodiment 1 is that: malononitrile:triethyl orthoformate:acetic anhydride=1:1.5:2.5 in molar ratio.

### Embodiment 6

A pump A system is malononitrile (100 g, 1.51 mol)+acetic anhydride (309.1 g, 3.03 mol), a pump B system is triethyl orthoformate (135.0 g, 9.08 mol), the speed of a pump A is: 24.8 g/min, and the speed of a pump B is: 15.7 g/min. The pump A and the pump B are simultaneously opened to pump two raw materials into a continuous reactor (the volume is 5 L), the temperature is controlled at 120° C., the outlet back pressure of the continuous reactor is 0.3-0.5 MPa, the residence time is 100 min, the outlet sampling and GC analysis are performed, and the remaining raw material is 0.0-0.5%. An effluent system is concentrated to 6 V, and 4 V of n-heptane is dropwise added for crystallization. After being filtered, 141.0 g of an ethoxymethylenemalononitrile product is obtained, the GC purity is 91.2%, and the yield is 69.6%.

A difference from Embodiment 1 is that: malononitrile:triethyl orthoformate:acetic anhydride=1:6.0:2.0 in molar ratio.

### Embodiment 7

A pump A system is malononitrile (100 g, 1.51 mol)+acetic anhydride (309.1 g, 3.03 mol), a pump B system is triethyl orthoformate (135.0 g, 9.08 mol), the speed of a pump A is: 24.8 g/min, and the speed of a pump B is: 15.7 g/min. The pump A and the pump B are simultaneously opened to pump two raw materials into a continuous reactor (the volume is 5 L), the temperature is controlled at 120° C., the outlet back pressure of the continuous reactor is 0.3-0.5 MPa, the residence time is 100 min, the outlet sampling and GC analysis are performed, and the remaining raw material is 0.0-0.5%. An effluent system is concentrated to 6 V, and 4 V of n-heptane is dropwise added for crystallization. After being filtered, 140.1 g of an ethoxymethylenemalononitrile product is obtained, the GC purity is 91.6%, and the yield is 68.7%.

A difference from Embodiment 1 is that: malononitrile:triethyl orthoformate:acetic anhydride=1:0.9:6.0 in molar ratio.

### Embodiment 8

A pump A system is malononitrile (100 g, 1.51 mol)+acetic anhydride (325 g, 3.18 mol), a pump B system is triethyl orthoformate (247 g, 1.67 mol), the speed of a pump A is: 24.8 g/min, and the speed of a pump B is: 15.7 g/min. The pump A and the pump B are simultaneously opened to pump two raw materials into a continuous reactor (the volume is 2.5 L), the temperature is controlled at 120° C., the outlet back pressure of the continuous reactor is 0.3-0.5 MPa, the residence time is 45 min, the outlet sampling and GC analysis are performed, and the remaining raw material is 16-17%. An effluent system is concentrated to 6 V, and 4 V of n-heptane is dropwise added for crystallization. After being filtered, 143.1 g of an ethoxymethylenemalononitrile product is obtained, the gas phase purity is 72.3%, and the yield is 67.96%.

A difference from Embodiment 1 is that: the residence time is 45 min.

### Embodiment 9

A pump A system is malononitrile (100 g, 1.51 mol)+acetic anhydride (325 g, 3.18 mol), a pump B system is triethyl orthoformate (247 g, 1.67 mol), the speed of a pump A is: 24.8 g/min, and the speed of a pump B is: 15.7 g/min. The pump A and the pump B are simultaneously opened to pump two raw materials into a continuous reactor (the volume is 5 L), the temperature is controlled at 120° C., the outlet back pressure of the continuous reactor is 0.3-0.5 MPa, the residence time is 90 min, the outlet sampling and GC analysis are performed, and the remaining raw material is 0.0-0.5%. An effluent system is concentrated to 6 V, and 4 V of n-heptane is dropwise added for crystallization. After being filtered, 182.3 g of an ethoxymethylenemalononitrile product is obtained, the GC purity is 99.5%, and the yield is 98.12%.

A difference from Embodiment 1 is that: the residence time is 90 min.

### Embodiment 10

A pump A system is malononitrile (100 g, 1.51 mol)+acetic anhydride (325 g, 3.18 mol), a pump B system is triethyl orthoformate (247 g, 1.67 mol), the speed of a pump A is: 24.8 g/min, and the speed of a pump B is: 15.7 g/min. The pump A and the pump B are simultaneously opened to pump two raw materials into a continuous reactor (the volume is 5 L), the temperature is controlled at 120° C., the outlet back pressure of the continuous reactor is 0.3-0.5 MPa, the residence time is 120 min, the outlet sampling and GC analysis are performed, and the remaining raw material is 0.0-0.5%. An effluent system is concentrated to 6 V, and 4 V of n-heptane is dropwise added for crystallization. After being filtered, 182.7 g of an ethoxymethylenemalononitrile product is obtained, the GC purity is 99.5%, and the yield is 98.33%.

A difference from Embodiment 1 is that: the residence time is 120 min.

### Embodiment 11

A pump A system is malononitrile (100 g, 1.51 mol)+acetic anhydride (325 g, 3.18 mol), a pump B system is triethyl orthoformate (247 g, 1.67 mol), the speed of a pump A is: 24.8 g/min, and the speed of a pump B is: 15.7 g/min. The pump A and the pump B are simultaneously opened to pump two raw materials into a continuous reactor (the volume is 2.5 L), the temperature is controlled at 120° C., the outlet back pressure of the continuous reactor is 0.3-0.5 MPa, the residence time is 30 min, the outlet sampling and GC analysis are performed, and the remaining raw material is 19-22%. An effluent system is concentrated to 6 V, and 4 V of n-heptane is dropwise added for crystallization. After being filtered, 139.4 g of an ethoxymethylenemalononitrile product is obtained, the gas phase purity is 70.8%, and the yield is 53.39%.

A difference from Embodiment 1 is that: the residence time is 30 min.

### Embodiment 12

A pump A system is malononitrile (100 g, 1.51 mol)+acetic anhydride (325 g, 3.18 mol), a pump B system is triethyl orthoformate (247 g, 1.67 mol), the speed of a pump A is: 24.8 g/min, and the speed of a pump B is: 15.7 g/min. The pump A and the pump B are simultaneously opened to pump two raw materials into a continuous reactor (the volume is 5 L), the temperature is controlled at 120° C., the outlet back pressure of the continuous reactor is 0.3-0.5 MPa, the residence time is 400 min, the outlet sampling and GC analysis are performed, and the remaining raw material is 0-0.5%. An effluent system is concentrated to 6 V, and 4 V of n-heptane is dropwise added for crystallization. After being filtered, 180.0 g of an ethoxymethylenemalononitrile product is obtained, the gas phase purity is 61.2%, and the yield is 59.59%.

A difference from Embodiment 1 is that: the residence time is 400 min.

### Embodiment 13

A pump A system is malononitrile (100 g, 1.51 mol)+acetic anhydride (325 g, 3.18 mol), a pump B system is triethyl orthoformate (247 g, 1.67 mol), the speed of a pump A is: 24.8 g/min, and the speed of a pump B is: 15.7 g/min. The pump A and the pump B are simultaneously opened to pump two raw materials into a continuous reactor (the volume is 5 L), the temperature is controlled at 110° C., the outlet back pressure of the continuous reactor is 10 MPa, the residence time is 100 min, the outlet sampling and GC analysis are performed, and the remaining raw material is 0.0-0.5%. An effluent system is concentrated to 6 V, and 4 V of n-heptane is dropwise added for crystallization. After being filtered, a solid is dried to obtain 180.4 g of an ethoxymethylenemalononitrile product, the GC purity is 98.9%, and the yield is 96.51%.

A difference from Embodiment 1 is that: the reaction temperature is 110° C., and the pressure is 10 MPa.

### Embodiment 14

A pump A system is malononitrile (100 g, 1.51 mol)+acetic anhydride (325 g, 3.18 mol), a pump B system is triethyl orthoformate (247 g, 1.67 mol), the speed of a pump A is: 24.8 g/min, and the speed of a pump B is: 15.7 g/min. The pump A and the pump B are simultaneously opened to pump two raw materials into a continuous reactor (the volume is 5 L), the temperature is controlled at 150° C., the outlet back pressure of the continuous reactor is 0.9 MPa, the residence time is 100 min, the outlet sampling and GC analysis are performed, and the remaining raw material is 0.0-0.5%. An effluent system is concentrated to 6 V, and 4 V of n-heptane is dropwise added for crystallization. After being filtered, 185.6 g of an ethoxymethylenemalononitrile product is obtained, the gas phase purity is 59.4%, and the yield is 59.64%.

A difference from Embodiment 1 is that: the reaction temperature is 150° C., and the pressure is 0.9 MPa.

It should be noted that, in the above embodiments, the continuous reaction device is operated continuously for 24×7 h, and a reaction column and the like is equipped with corresponding standby devices and parts, it may achieve the on-line replacement of the devices without stopping, as to achieve a fully continuous reaction without interruption in the whole process.

The above are only preferred embodiments of the present disclosure, and are not intended to limit the present disclosure. For those skilled in the art, the present disclosure may have various modifications and changes. Any modifications, equivalent replacements, improvements and the like made within the spirit and principle of the present disclosure shall be included within a scope of protection of the present disclosure.

1. A continuous synthesis method of ethoxymethylenemalononitrile, comprising the following steps: continuously introducing malononitrile, triethyl orthoformate, and acetic anhydride into a continuous reaction device for condensation reaction to obtain the ethoxymethylenemalononitrile, and continuously discharging the generated ethoxymethylenemalononitrile; wherein the molar ratio of the malononitrile, the triethyl orthoformate and the acetic anhydride is 1:(0.9-6.0):(2.0-6.0).

2. The continuous synthesis method of ethoxymethylenemalononitrile according to claim 1, wherein the molar ratio of the malononitrile, the triethyl orthoformate and the acetic anhydride is 1:(1.1-1.5):(2.1-2.5).

3. The continuous synthesis method of ethoxymethylenemalononitrile according to claim 1, wherein the reaction temperature of the condensation reaction is 110 to 150° C., and a reaction pressure is 0.3 to 10 MPa.

4. The continuous synthesis method of ethoxymethylenemalononitrile according to claim 3, wherein the reaction temperature of the condensation reaction is 110 to 120° C., and the reaction pressure is 0.3 to 10 MPa.

5. The continuous synthesis method of ethoxymethylenemalononitrile according to claim 1, wherein the condensation reaction is carried out without or with a solvent.

6. The continuous synthesis method of ethoxymethylenemalononitrile according to claim 5, wherein when a solvent is used, the solvent is selected from one or more of ethyl acetate, tetrahydrofuran, 2-methyltetrahydrofuran, 1,4-dioxane, ethylene glycol dimethyl ether, diethylene glycol dimethyl ether, benzene, toluene, xylene, acetone, acetonitrile, methanol, ethanol, isopropanol, and ethylene glycol.

7. The continuous synthesis method of ethoxymethylenemalononitrile according to claim 5, wherein during the condensation reaction, residence time of the malononitrile, the triethyl orthoformate and the acetic anhydride in the continuous reaction device is 30 to 400 min.

8. The continuous synthesis method of ethoxymethylenemalononitrile according to claim 7, wherein during the condensation reaction, residence time of the malononitrile, the triethyl orthoformate and the acetic anhydride in the continuous reaction device is 90 to 120 min.

9. The continuous synthesis method of ethoxymethylenemalononitrile according to claim 1, wherein the continuous reaction device is a tubular reactor or a columnar reactor.

10. The continuous synthesis method of ethoxymethylenemalononitrile according to claim 5, wherein during the condensation reaction, the malononitrile, the triethyl orthoformate, the acetic anhydride, and the optional solvent are pumped into the continuous reaction device, and the total flowrate is 20 mL/min to 5 L/min.

11. The continuous synthesis method of ethoxymethylenemalononitrile according to claim 2, wherein the condensation reaction is carried out without or with a solvent.

12. The continuous synthesis method of ethoxymethylenemalononitrile according to claim 3, wherein the condensation reaction is carried out without or with a solvent.

13. The continuous synthesis method of ethoxymethylenemalononitrile according to claim 4, wherein the condensation reaction is carried out without or with a solvent.

14. The continuous synthesis method of ethoxymethylenemalononitrile according to claim 2, wherein the continuous reaction device is a tubular reactor or a columnar reactor.

15. The continuous synthesis method of ethoxymethylenemalononitrile according to claim 3, wherein the continuous reaction device is a tubular reactor or a columnar reactor.

16. The continuous synthesis method of ethoxymethylenemalononitrile according to claim 4, wherein the continuous reaction device is a tubular reactor or a columnar reactor.
